(19) United States
(12) Patent Application Publication
(10) Pub. No.: US 2025/0366730 A1
(43) Pub. Date: Dec. 4, 2025

(54) LOCATING BAR AND PATIENT SUPPORT SYSTEM

(71) Applicant: Elekta AB (publ), Stockholm (SE)

(72) Inventors: Gunnar Backman, Crawley (GB); Henrik Alexis, Crawley (GB); Kjell Eldered, Crawley (GB); Maja Nilsson, Crawley (GB)

(21) Appl. No.: 19/298,897

(22) Filed: Aug. 13, 2025

Related U.S. Application Data
(63) Continuation of application No. 16/242,769, filed on Jan. 8, 2019.
(60) Provisional application No. 62/614,518, filed on Jan. 8, 2018.

(51) Int. Cl. A61B 5/055; A61B 6/04; A61G 7/012; A61G 7/05

(52) U.S. Cl. CPC A61B 5/055; A61B 6/0407; A61G 7/05; A61G 7/012; A61G 2210/50

(57) ABSTRACT

A system includes a patient support including an elongate patient support surface; a pair of locating tracks, each locating track having an inward facing surface, and an outward facing surface in which a series of spaced grooves are defined, the locating tracks being arranged such that a space for a locating bar is defined between the inward facing surfaces, and respective grooves of the outward facing surfaces of the pair of locating tracks are aligned with each other; and a locating bar. The locating bar comprises: a substantially rigid bar structure dimensioned to fit between the inward facing surfaces of the locating tracks; and an end fitting provided at each of opposing ends of the bar structure, each end fitting including an abutment surface, and a resilient locking formation configured to engage in one of the grooves of the respective locating track.

### FIELD OF THE INVENTION

This application claims priority to U.S. Provisional Patent Application 62/214,518 filed Jan. 8, 2018, the contents of which are hereby incorporated by reference.

The present disclosure relates to a locating bar and a system incorporating a locating bar for supporting a patient in a radiotherapy system, computed tomography scan, or magnetic resonance imaging.

### BACKGROUND ART

Patient supports for use in radiotherapy systems are disclosed in US 2009/0013468. Such systems can be used with image guided radiotherapy systems of the type disclosed in U.S. Pat. No. 9,694,205. One requirement of patient support systems is that various equipment can be located on the support so as to hold the patient in a predetermined position for treatment.

One locating system comprises a bar having pegs at its ends, the pegs being engagable in holes in the patient support surface, and providing a means by which equipment can be located on the support surface. It is often necessary to reposition the bar to accommodate different patients. Any such system must therefore hold the equipment securely in place and be reliably repositionable to a new location.

Because it is sometimes necessary for the patient support to be useable with a Magnetic Resonance Imaging (MRI) system, the system for locating the bar must also allow positioning of coil arrangements forming part of the MRI system.

The present disclosure seeks to address one or more of these requirements.

### SUMMARY

A first aspect of the disclosure provides a locating bar for use with a patient support, comprising an elongate patient support surface having a pair of locating tracks defined along opposite long edges of the support surface, each locating track having an inward facing surface, and an outward facing surface in which a series of spaced grooves are defined, the locating tracks being arranged such that a space for the locating bar is defined between the inward facing surfaces, and respective grooves of the outward facing surfaces of the pair of locating tracks are aligned with each other; the locating bar comprising: a substantially rigid bar structure dimensioned to fit between the inward facing surfaces of the locating tracks so as to lie transverse to a longitudinal axis of the support surface; and an end fitting provided at each of opposing ends of the bar structure, each of the end fittings including an abutment surface configured to engage the inward facing surface of a respective locating track, and a resilient locking formation configured to engage in one of the grooves in the respective locating track.

In accordance with some embodiments, the locating bar can further comprise at least one locating formation, e.g. a pair of locating formations, projecting upwardly from the bar structure and configured to engage in a corresponding locating recess in a piece of equipment to be positioned on the upper surface of the patient support. The locating formations can be conical in shape.

In accordance with some embodiments, the end fitting can comprise an upper member extending from the end surface and configured to project over the respective locating track, and a locking formation extending downwardly from the upper member. The upper member can include a tab projecting beyond the locking formation for moving the locking formation out of engagement with the groove of the locating track.

In accordance with some embodiments, the end fitting can be formed from a resilient plastic material.

Another aspect of the disclosure provides a system, comprising: a patient support including an elongate patient support surface; a pair of locating tracks defined along opposite long edges of the support surface, each locating track having an inward facing surface, and an outward facing surface in which a series of spaced grooves are defined, the locating tracks being arranged such that a space for a locating bar is defined between the inward facing surfaces, and respective grooves of the outward facing surfaces of the pair of locating tracks are aligned with each other; and a locating bar comprising: a substantially rigid bar structure dimensioned to fit between the inward facing surfaces of the locating tracks so as to lie transverse to a longitudinal axis of the support surface; and an end fitting provided at each of opposing ends of the bar structure, each of the end fittings including an abutment surface configured to engage the inward facing surface of a respective one of the locating tracks, and a resilient locking formation configured to engage in one of the grooves of the respective locating track.

In accordance with some embodiments, the system can further comprise a pair of guide tracks located outside the locating tracks in which a coil support stand can be secured so as to be slidable with respect to the patient support surface.

In accordance with some embodiments, the system can further comprise a series of indexing marks adjacent each locating track.

A further aspect of the disclosure provides a system, comprising: a patient support including an elongate patient support surface; a first pair of locating tracks defined along opposite long edges of the support surface, each locating track having an inward facing surface, and an outward facing surface in which a series of spaced grooves are defined, the locating tracks being arranged such that a space for a locating bar is defined between the inward facing surfaces, and respective grooves of the outward facing surfaces of the pair of locating tracks are aligned with each other; a second pair of locating tracks located outboard of the first pair of locating tracks and configured to slidably receive a support stand; and a locating bar comprising: a substantially rigid bar structure dimensioned to fit between the inward facing surfaces of the locating tracks so as to lie transverse to a longitudinal axis of the support surface; and an end fitting provided at each of opposing ends of the bar structure, each of the end fittings including an abutment surface configured to engage the inward facing surface of a respective one of the locating tracks, and a resilient locking formation configured to engage in one of the grooves in the respective locating track.

In accordance with some embodiments, at least one coil can be mounted on the support stand, the at least one coil being configured to operate as part of a magnetic resonance imaging system.

In accordance with some embodiments, the support stand can include a height adjustment mechanism by which the height of the at least one coil can be adjusted.

In accordance with some embodiments the patient support, the first and second locating tracks, and the at least one locating bar can be made from non-metallic materials.

A further aspect of the disclosure provides a method, comprising: providing a patient support including an elongate patient support surface, a pair of locating tracks defined along opposite long edges of the support surface, each locating track having an inward facing surface, and an outward facing surface in which a series of spaced grooves are defined, the locating tracks being arranged such that a space for a locating bar is defined between the inward facing surfaces, and respective grooves of the outward facing surfaces of the pair of locating tracks are aligned with each other; providing a locating bar including a substantially rigid bar structure dimensioned to fit between the inward facing surfaces of the locating tracks so as to lie transverse to a longitudinal axis of the support surface, and an end fitting provided at each of opposing ends of the bar structure; and engaging an abutment surface of each end fitting with the inward facing surface of a respective one of the locating tracks, while engaging a resilient locking formation of each end fitting in one of the grooves of the respective locating track to secure the locating bar to the patient support surface using the locking formation.

### BRIEF DESCRIPTION OF THE DRAWINGS

Embodiments of the disclosure will now be described by way of example and with reference to the accompanying figures.

FIG. 1 is a schematic view of a patient support system for use with an MRI-guided radiotherapy system;

FIG. 2 is a more detailed partial view of the patient support surface illustrated in FIG. 1;

FIG. 3a is a perspective view of a locating bar of the patient support system illustrated in FIG. 1;

FIG. 3b is a bottom view of the locating bar illustrated in FIG. 3a;

FIG. 3c is an exploded view of the locating bar illustrated in FIG. 3a;

FIG. 4a is a top view of the locating bar secured to a patient support surface of the patient support system illustrated in FIG. 1;

FIG. 4b is a more detailed view of an end fitting of the locating bar illustrated in FIG. 4a.

FIG. 5 is an alternative embodiment of the end fitting illustrated in FIG. 4b;

FIGS. 6a and 6b illustrate a method for securing the locating bar to the patient support surface according to an embodiment of the disclosure.

### DETAILED DESCRIPTION

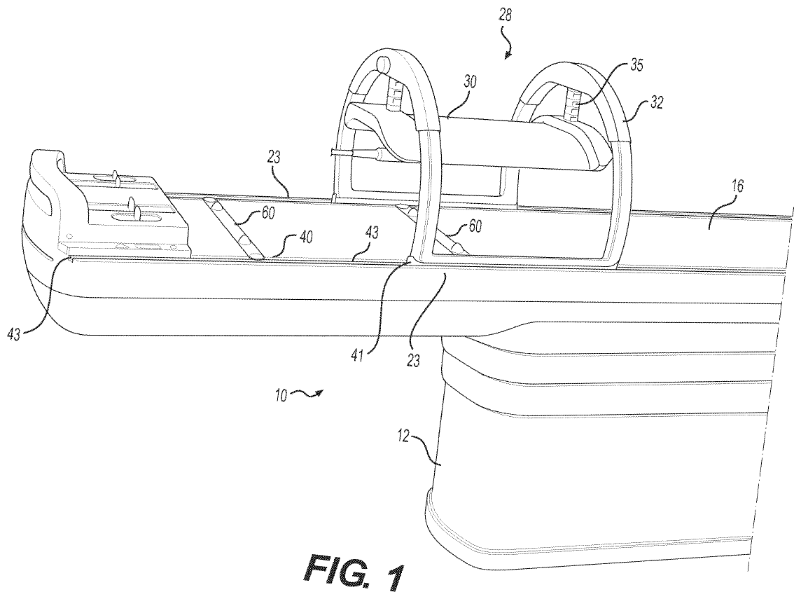

FIG. 1 shows a schematic view of a patient support system 10, such as

that described in US 2018/0289283 (incorporated herein by reference), for use with an image guided radiotherapy system of the type disclosed in U.S. Pat. No. 9,694,205 or Computed Tomography. The system 10 comprises a patient support 12, and a patient support surface 16 which is movable between the patient support 12 and a treatment table (not shown). The patient support 12 comprises a mechanism for altering the height of the patient support surface 16.

An MRI coil arrangement 28 can be arranged on the system 10 as is shown in FIG. 1. The MRI coil arrangement 28 includes an anterior coil 30 and a support stand 32. Support stand 32 includes rails 41 configured to be slidably received in grooves or guide tracks 43 provided adjacent longitudinal edges 23 of patient support surface 16. The coil arrangement 28 may further comprise a height adjustment mechanism 35 for adjusting the height of the anterior coil 30 above the patient support surface 16. The height adjustment mechanism 35 may enable an operator to adjust the height of the anterior coil 30 in a direction perpendicular to a plane defined by the patient support surface 16 or in a direction oblique to the plane defined by the patient support surface 16 (by means not shown). A pair of movable locating bars 60 are positioned on patient support surface 16 and connected to the locating tracks 40 as is described in further detail below.

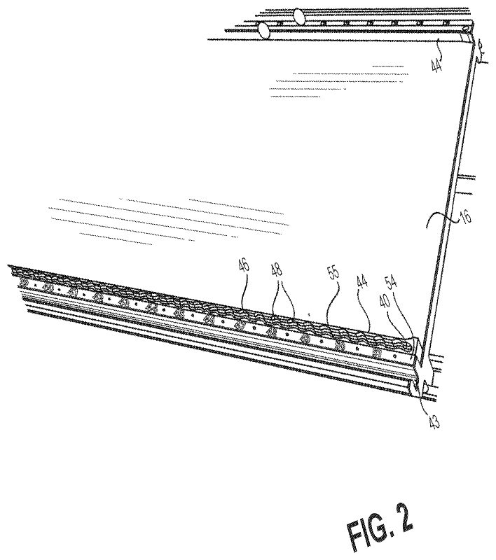

FIG. 2 is a more detailed partial view of the patient support surface 16. The patient support surface 16, which may be configured as a bed, couch or support pad, is a substantially planar elongate support surface. A locating track 40 extends along each edge 23 of the bed 16. Each locating track is formed of an upstanding ridge having a relatively smooth inward facing surface 44 and an outward facing surface 46 that has a series of regularly spaced grooves 48. The locating tracks 40 are positioned such that the grooves 48 are substantially aligned across the bed 16. The guide tracks 43 are located outboard of the locating tracks 40, and index marks or numbers 55 are provided on an upper surface of a ridge 54 extending between the locating and the guide tracks 40, 43. One having ordinary skill in the art will appreciate that index marks 55 may include any of a variety of visual markings suitable for alerting the user to the position of each locating bar 60 with respect to the tracks 40. Such markings include but are not limited to: ticks, shapes, grooves, numbers, letters etc. and may be uniform or non-uniform in color.

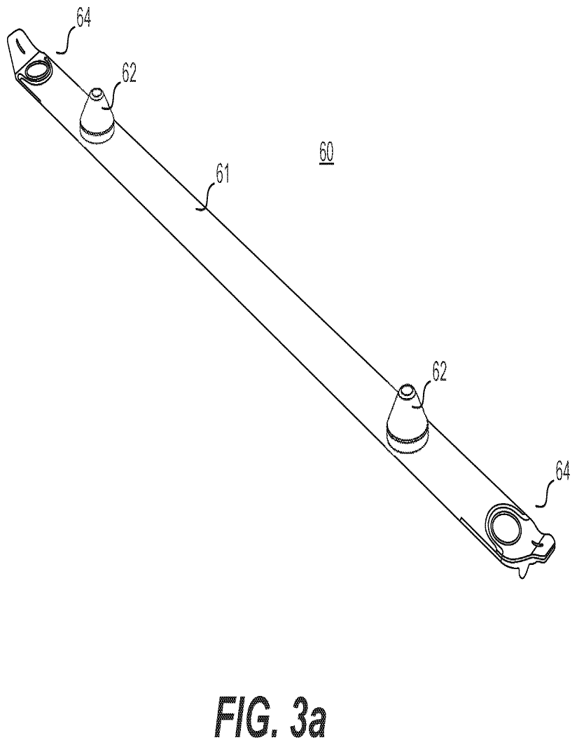

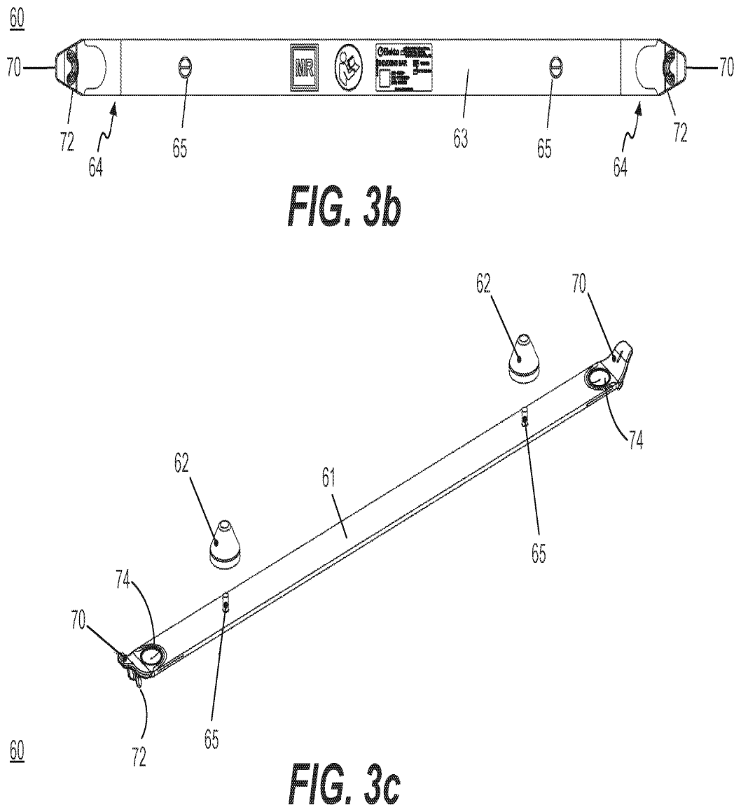

FIGS. 3a-3c illustrate various views of the locating bar 60. The locating bar 60 includes a substantially rigid, rectangular non-metal bar 61 having opposite end portions. Each end portion may be fitted with (or alternatively define) an end fitting 64 having an upper member 68 and an abutment surface 66, the details of which will be further described below. As illustrated in FIG. 3c, the locating bar 60 may further include a securing mechanism 65 for securing conical locating pegs 62 to an upper surface of the bar 61. In one embodiment, the securing mechanism 65 is a screw for screwing into corresponding threaded holes (not shown) in the locating pegs 62; however, persons having ordinary skill in the art will understand that the locating pegs 62 may be secured to the bar 61 using an interference fit or any of a variety of mechanisms suitable for securing the pegs 62 to the bar 61. Alternatively, the locating pegs 62 may be permanently affixed to the bar 61. A bottom surface 63 of locating bar 60 may include various warnings and labels to ensure proper use and handling, as illustrated in FIG. 3b.

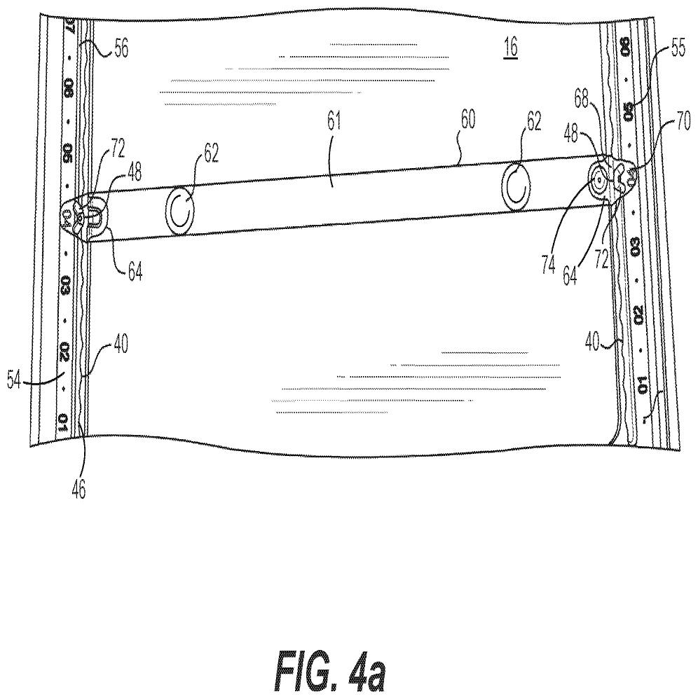

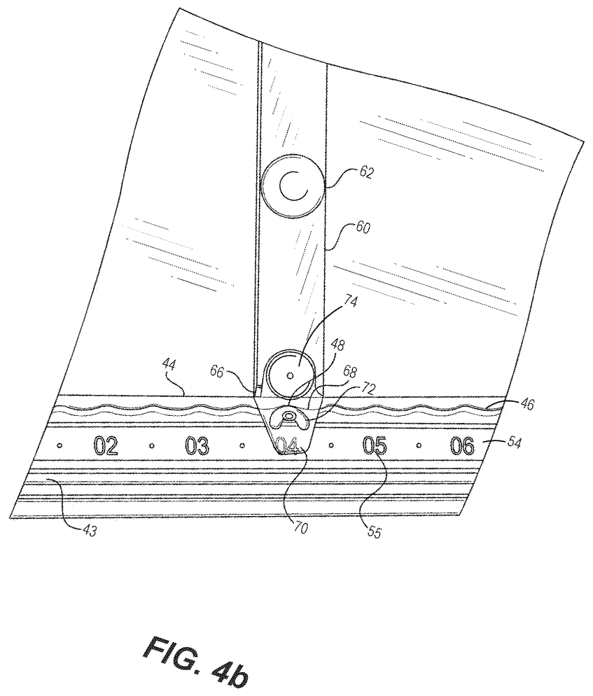

FIGS. 4a and 4b illustrate the locating bar 60 positioned on the bed 16. In use, one or more locating bars 60 extend transversely across the surface of the bed 16 between the locating tracks 40. The dimensions of the bar 60 and the end fittings 64 are such that the locating bar 60 is constrained to extend substantially at right angles to a longitudinal axis of the bed 16. The pegs 62 are configured to secure to various pieces of equipment (not shown) which allow the patient to be supported in a predetermined position for treatment. Locating pegs 62 may fit into corresponding locating holes in the equipment or be otherwise affixed to the equipment. Alternatively, pegs 62 may include one or more locating holes for receiving one or more pegs disposed on the equipment.

As shown in FIG. 4a, a side 56 of the ridge 54 facing the outward facing surface 46 is vertically inclined such that a channel formed between the outward facing surface 46 and the side 56 is wider at the top than at the bottom. This tapered channel facilitates positioning of the end of the end fittings 64 of each locating bar 60. The base of the outward facing surface 46 can be undercut so that the end fitting 64 is prevented from accidental vertical disengagement when a locking formation 72 is engaged in a groove 48.

FIG. 4b illustrates various details of the end fitting 64. The end fitting 64 may be made of a transparent resilient plastic material. The upper member 68 terminates in a tab 70 and includes a locking formation 72 extending from a bottom surface thereof. The locking formation 72 is complementary in shape to the groove 48 defined in each locating track 40. In use, the abutment surface 66 bears against the inward facing surface 44 of the locating track 40 and the upper member 68 extends over the locating track 40. The dimensions and resiliency of the end fitting 64 are such that the locking formation 72 is urged into the groove 48 by the upper member 68.

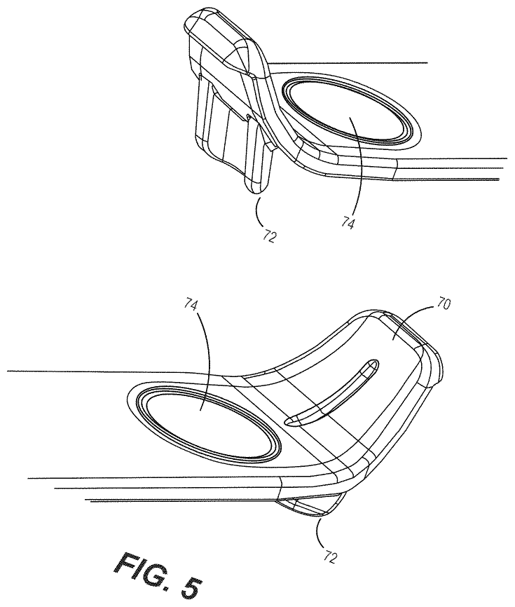

FIG. 5 illustrates an alternative embodiment in which the upper portion 68 may be disposed at a steeper angle with respect to an upper surface of the bar 61 to facilitate placement of the locking formation 72. The locating bar 60 can be configured such that it is not possible for the locking formations 72 to fully engage in grooves 48 that are not aligned across the bed 16, i.e., with the bar at an angle to the transverse direction.

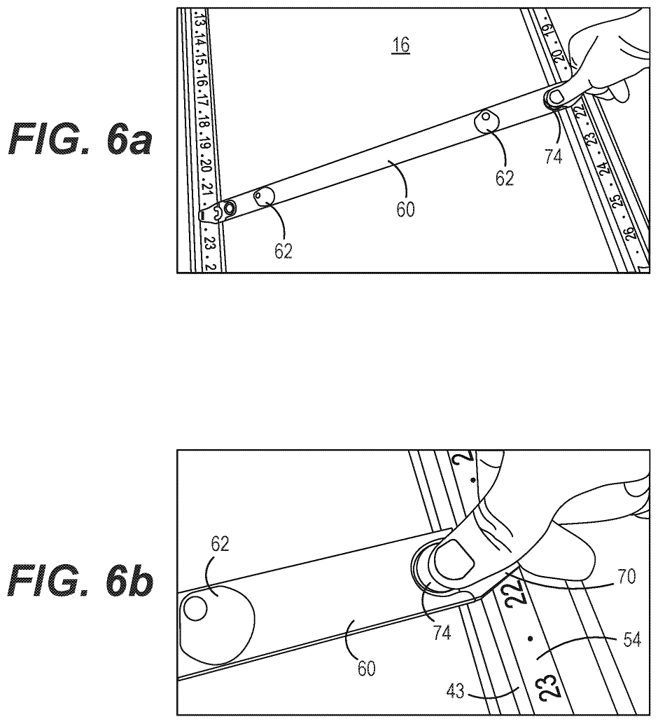

Methods for positioning the locating bar 60 are described next. In use, the position of the locating bar 60 can be adjusted by raising the tabs 70 to disengage the locking formations 72 from respective grooves 48. Once the locking formations 72 are disengaged, the locating bar 60 is free to slide along the surface of the bed 16 and may be moved along locating tracks 40 to a desired position defined by index marks 55. Upon reaching the desired position, the tabs 70 are resiliently released to allow the locking formations 72 to engage in respective grooves 48 and lock the locating bar 60 in position. In one embodiment, end fittings 64 further comprise a pressure pad 74. As illustrated in FIGS. 6a and 6b, a user may place a finger (e.g. thumb) on the pressure pad 74 and another finger (e.g. index finger) on the underside of the tab 70 and press the pad while holding the tab to insert the locking formation 72 into a desired groove 48. The resiliency of the locking formation 72 allows it to be inserted into the groove 48 and to subsequently click into place. The click provides audible confirmation that the locking formation 72 has been properly placed in the groove 48. The end fittings 64 may be inserted simultaneously, e.g., with one hand guiding each end fitting, or sequentially. When the end fittings 64 are inserted sequentially, the user may lightly secure a first end fitting 64 at a far end of the locating bar 60 by placing the locking formation 72 in a desired groove 48 located opposite the user and pulling the locating bar 60 towards the user, and then securing a second end fitting 64 at a near end of the locating bar 60 by placing the locking formation 72 in a corresponding desired groove 48 located near the user. These steps may be performed in reverse to remove the locating bar 60. The transparency of the end fittings 64 allow the marks 55 to be viewed through the tabs 70. With the locating bar 60 locked in place, a suitable piece of equipment, such as a head rest or limb support (not shown) can be secured to the locating pegs 62. Thereafter, the patient may be placed on the bed 16 and supported as necessary by the equipment. The bed 16 is then advanced into the radiotherapy system to commence patient treatment.

Where the bed is to be used with an MRI system, the MRI coil support 28 shown in FIG. 1 can be positioned on the bed, with the rails 41 engaged in the guide tracks 43. The support 28 may be slid along the bed so that the anterior coil 30 is positioned over the patient at the required treatment position. Since the guide tracks 43 are outboard of the locating tracks 40, the coil support 28 can be moved over the patient without interfering with the positioning of the locating bar 60. Due to the high magnetic fields used in MRI systems, system components disclosed herein are constructed of non-metallic materials.

It will be understood that persons having ordinary skill in the art may make various alterations and changes without departing from the essence and scope of the present disclosure. The shape and spacing of the locating pegs 62 may be adapted to suit the design of the equipment or the needs of the patient. For example, the locating pegs 62 may be configured in various shapes and may be repositioned along the length of the bar. If the locating pegs are repositionable, the bar 61 may be fitted with numerous securing mechanisms 65 to allow the user to vary the position of the locating pegs 62 along the bar 61, or the securing mechanisms 65 may be repositioned along the length of the bar 61 to enable various peg positions. Repositionable securing mechanisms 65 may be slidable and securable along a track (not shown) defined along a length of the bar or fitted into discrete recesses (not shown) defined along the length of the bar 61. The shape and spacing of the locating pegs 62 may also be selected to be incompatible with non-MRI compliant equipment so as to prevent such equipment from being incorrectly used with the MRI system. Furthermore, the exact shape and size of the bar 61 and the dimensions and profiles of the grooves 48 and locking formations 72 can be selected in accordance with the preferences of the user and/or needs of the patient. The various components may also be designed to be easily cleaned and reusable. Materials may be selected to enhance strength and thus improve wear and tear resistance. Accordingly, the disclosure is not limited by embodiments disclosed herein and the scope of protection should be that as defined by the claims.

1.-20. (canceled)

21. A patient support system comprising:
a patient support surface;
a first track system disposed along opposite edges of the patient support surface, the first track system including spaced indexing positions;
a positioning bar configured to engage with the first track system, the positioning bar configured to be operated from one side of the patient support system, the positioning bar comprising:
a bar structure;
an at least partially transparent portion disposed at or adjacent to an end portion of the positioning bar; and
an engagement element disposed at or adjacent to the end portion of the positioning bar, the engagement element configured to releasably secure the positioning bar to selected indexing positions of the first track system, and
indexing marks provided adjacent to the first track system, the indexing marks configured to provide guidance on a position of the selected indexing positions of the positioning bar relative to the first track system, the indexing marks visible via the at least partially transparent portion of the positioning bar.

22. The patient support system of claim 21, wherein the first track system comprises a pair of locating tracks, a locating track of the pair of locating tracks including an inward facing surface and an outward facing surface, and wherein the spaced indexing positions include a series of grooves defined in the outward facing surface.

23. The patient support system of claim 22, wherein the engagement element includes a locking formation configured to engage with the grooves.

24. The patient support system of claim 23, wherein the engagement element includes an abutment surface configured to engage the inward facing surface of a respective locating track.

25. The patient support system of claim 23, wherein the engagement element includes a tab extendable beyond the locking formation to disengage the locking formation from a respective groove.

26. The patient support system of claim 25, wherein the engagement element includes a pressure pad disposed on the at least partially transparent portion, the pressure pad configured to receive finger pressure during engagement or disengagement of the locking formation.

27. The patient support system of claim 23, wherein the locking formation is configured to fully engage only when the positioning bar is substantially perpendicular to a longitudinal axis of the patient support surface.

28. The patient support system of claim of claim 21, wherein a second track system is disposed along the patient support surface and configured to receive a support stand independently of the first track system, and the second track system is disposed outboard of the first track system relative to a longitudinal centerline of the patient support surface.

29. The patient support system of claim 28, comprising a coil support stand slidably engaged with the second track system, the coil support stand including a coil configured to operate as part of a magnetic resonance imaging system.

30. The patient support system of claim 21, comprising a locating formation projecting upwardly from the bar structure, the locating formation configured to engage with a corresponding locating recess in equipment to be positioned on the patient support surface.

31. The patient support system of claim 30, wherein the locating formation includes a pair of conical pegs.

32. The patient support system of claim 30, wherein the locating formation is repositionable along a length of the bar structure.

33. The patient support system of claim 21, wherein the bar structure is substantially rigid and dimensioned to lie transverse to a longitudinal axis of the patient support surface.

34. The patient support system of claim 29, wherein the coil support stand includes a height adjustment mechanism.

35. A positioning bar for use with a patient support system, the positioning bar comprising:
a bar structure configured to extend across a patient support surface to enable the positioning bar to be operated from one side of the patient support system;
an at least partially transparent portion disposed at an end portion of the positioning bar, the at least partially transparent portion configured to permit visibility of indexing marks adjacent to a track system of the patient support surface; and
an engagement element disposed at the end portion of the positioning bar, the engagement element configured to releasably secure the positioning bar to selected indexing positions of the track system.

36. The positioning bar of claim 35, wherein the engagement element comprises:
a locking formation configured to engage with grooves in the track system; and
an abutment surface configured to engage an inward facing surface of the track system.

37. The positioning bar of claim 36, wherein the engagement element further comprises a tab extendable beyond the locking formation to disengage the locking formation from a respective groove.

38. The positioning bar of claim 37, wherein the engagement element further includes a pressure pad disposed on the at least partially transparent portion, the pressure pad configured to receive finger pressure during engagement or disengagement of the locking formation.

39. The positioning bar of claim 35, further comprising a locating formation projecting upwardly from the bar structure, the locating formation configured to engage with a corresponding locating recess in equipment to be positioned on the patient support surface.

40. The positioning bar of claim 39, wherein the locating formation comprises a pair of conical pegs, and wherein the locating formation is repositionable along a length of the bar structure.
